(19) United States
(12) Patent Application Publication
(10) Pub. No.: US 2020/0108069 A1
(43) Pub. Date: Apr. 9, 2020

(54) Treatment of Fibrotic Conditions

(71) Applicant: ANGLIA RUSKIN UNIVERSITY HIGHER EDUCATION CORPORATION, Chelmsford (GB)

(72) Inventors: Selim CELLEK, Chelmsford (GB); Marcus ILG, Chelmsford (GB); David RALPH, Ley Hill (GB); Asif MUNEER, Rickmansworth (GB); Marta MATEUS, Haverhill (GB); William STEBBEDS, Haverhill (GB)

(21) Appl. No.: 16/576,653

(22) Filed: Sep. 19, 2019

(30) Foreign Application Priority Data
Oct. 4, 2018 (GB) 1816215.6

(51) Int. Cl. A61K 31/5025; A61K 31/138; A61P 15/00

(52) U.S. Cl. CPC A61K 31/5025; A61P 15/00; A61K 31/138

(57) ABSTRACT

This invention relates to the treatment of fibrotic conditions, such as Peyronie's disease, by administering a phosphodiesterase type 5 (PDE5) inhibitor, such as vardenafil, and a selective oestrogen receptor modulator (SERM), such as tamoxifen, to an individual in need thereof.

### CROSS-REFERENCING

This application claims benefit of priority to United Kingdom Application No. 1816215.6, filed on Oct. 4, 2018, which applications incorporated by reference herein.

### FIELD

The present invention relates to the treatment of fibrotic conditions, such as Peyronie's disease.

### BACKGROUND

Fibrotic conditions are associated with the pathological deposition of excessive fibrous connective tissue. For example, Peyronie's disease is a fibrotic condition characterized by the formation of a fibrous plaque in the connective tissue surrounding the penile erectile tissue, the tunica albuginea (TA). It is a benign condition of unknown aetiology characterized by the formation of localized fibrous plaques, resulting in a penile deformity, manifesting as a curvature, indentation or shortening during erection. The disease has been shown to be prevalent, especially as men get older¹, and affects quality of life, principally through pain during erection, erectile dysfunction, loss of penetrative ability during intercourse and associated psychological stress^{2,3}. Despite the progress in understanding the pathophysiology of PD, there is currently a lack of efficacious medical therapies for PD, with surgery or collagenase injections⁴ being the main treatment options. Although the surgical outcomes following penile straightening are well documented, surgery is invasive, costly and is frequently detrimental to penile size and erectile functions.

Myofibroblasts have the features of both fibroblasts and smooth muscle cells and are characterized by the presence of alpha-smooth muscle actin (α-SMA) positive cytoplasmic fibres. These actin fibres contribute to the contractile ability of these cells⁶. Although various progenitor cells for myofibroblasts have been suggested⁷, they most often differentiate from locally residing fibroblasts through normal wound healing signalling, particularly, transforming growth factor-β1 (TGF-β1)⁸. Myofibroblasts have been shown to play vital and ubiquitous roles in both normal wound healing and fibrosis. Most important functions include production and remodelling of extracellular matrix (ECM) protein^{6,9}, and the secretion of profibrotic and pro-inflammatory cytokines⁹. These cells have been shown to be present in liver¹⁰, lung¹¹, kidney¹² and cardiac⁴³ fibrosis, as well as PD plaques^{13, 14}. It is therefore generally agreed that myofibroblasts play a critical role in the pathophysiology of fibrosis and the inhibition of myofibroblast transformation has been shown to be effective in preventing fibrosis¹⁵

### SUMMARY

The present inventors have discovered that phosphodiesterase type 5 inhibitors (PDE5i) exhibit an unexpected synergy with selective oestrogen receptor modulators (SERM) in the inhibition of fibrosis and the treatment of fibrotic conditions.

A first aspect of the invention provides a method of treatment of a fibrotic condition comprising administering a phosphodiesterase type 5 (PDE5) inhibitor and a selective oestrogen receptor modulator (SERM) to an individual in need thereof.

Fibrotic conditions may include Peyronie's disease.

A second aspect of the invention provides a method of reducing, inhibiting or preventing fibrosis comprising administering a phosphodiesterase type 5 (PDE5) inhibitor and a selective oestrogen receptor modulator (SERM) to an individual in need thereof.

A third aspect of the invention provides a method of inhibiting myofibroblast transformation and/or reducing the formation of excess fibrous connective tissue comprising administering a phosphodiesterase type 5 (PDE5) inhibitor and a selective oestrogen receptor modulator (SERM) to an individual in need thereof.

A fourth aspect of the invention provides a combination of a phosphodiesterase type 5 (PDE5) inhibitor and a selective oestrogen receptor modulator (SERM) for use in a method of any of the first, second or third aspects.

A fifth aspect of the invention provides the use of a combination of a phosphodiesterase type 5 (PDE5) inhibitor and a selective oestrogen receptor modulator (SERM) in the manufacture of a medicament for use in a method of any of the first, second or third aspects.

A sixth aspect of the invention provides a pharmaceutical composition comprising a phosphodiesterase type 5 (PDE5) inhibitor and a selective oestrogen receptor modulator (SERM). The composition may be used in a method of any of the first, second or third aspects.

PDE5 inhibitors that may be used in accordance with any of the above aspects of the invention include vardenafil.

SERMs that may be used in accordance with any of the above aspects of the invention include tamoxifen.

Other aspects and embodiments of the invention are described in more detail below.

### BRIEF DESCRIPTION OF THE FIGURES

The patent or application file contains at least one drawing executed in color. Copies of this patent application publication with color drawing(s) will be provided by the U.S. Patent and Trademark Office upon request and payment of the necessary fee.

FIG. 1 shows that TGF-β1 induces transformation of fibroblasts that were isolated from human tunica albuginea (TA) to myofibroblasts. The transformation can be measured using a high throughput screening assay. Fibroblasts were exposed to TGF-β1 (10 ng/ml) for 72 hours. Representative images of α-SMA staining in (1A) untreated TA-derived cells and (1B) TA-derived cells exposed to TGF-β1. Images were captured at 200× magnification. Scale bars 50 μm. FIG. 1C shows quantification of α-SMA positive cells. FIG. 1D shows the mRNA levels of α-SMA were determined using the 2^{-ΔΔCt} method. Data points plotted as mean±SEM, N=3 patients for each group; n=9. *P<0.05 vs negative control. FIG. 1E shows representative Western blot for α-SMA content in protein lysates from untreated cells and cells exposed to 10 ng/ml TGF-β1 for 72 h: 20 μg of protein was loaded under reducing conditions. Lower bands (35 kD) represent GAPDH loading control, higher bands (42 kD) represent α-SMA. Lane 1: protein ladder, Lanes 2, 4, 6, 8: untreated TA derived cells. Lanes 3, 5, 7, 9: cells exposed to 10 ng/ml TGF-β1. FIG. 1F shows statistical validation of the ICE method. Positive controls correspond to wells exposed to TGF-β1, negative controls correspond to wells exposed to media only. Data normalized to nuclear dye intensity. Validation for high throughput screening by calculation of Z′ comparing negative control wells to positive control wells, yielding a Z′ value of 0.89.

FIG. 2 shows concentration response curves for hits acquired from screening campaign. Effect of PDE5i vardenafil, sildenafil, and tadalafil (2A) and SERMs tamoxifen and raloxifen (2B) on TGF-β1-induced myofibroblasts transformation. Cells derived from TA tissue were exposed to a range of concentrations of PDE5 is between 0.03 and 100 μM in co-incubation with 10 ng/ml TGF-β1 for 72 hours. Data points were plotted as average±SEM of the percentage of maximum response of the α-SMA/DNA staining ratio, N=3; n=9.

FIG. 3 shows the effect of compounds on TGF-β1-induced myofibroblast collagen contraction in fibroblast populated collagen lattices (FPCL). FIG. 3A shows representation of the contraction of the fibroblast populated collagen lattices. Top row: example for no contraction after release. Bottom row: example for uniform contraction after release from wall of the well. FPCLs were exposed to 10 ng/ml TGF-β1 and various concentrations of tamoxifen (3B) or vardenafil (3C). FPCLs were released after 72 h and contraction was observed for 8 h. Data presented as percentage of maximum collagen contraction compared to vehicle control (DMSO) in cells exposed to tamoxifen/vardenafil. Data points were plotted as mean±SEM, N=3; n=9. *P<0.05 vs vehicle control at the same time point.

FIG. 4 shows the effect of compounds on TGF-β1-induced myofibroblast ECM production. FIG. 4A shows cells derived from TA tissue were exposed to a range of concentrations of vardenafil, tamoxifen or SB-505124 in co-incubation with 10 ng/ml TGF-β1 for 7 days. ECM was stained for collagen I (Col I; 4A); collagen III (Col III; 4B); collagen V (Col V; 4C) and fibronectin (4D) after cell lysis. Data points were plotted as average±SEM of the percentage of maximum response of protein/pre-lysis DNA staining ratio, N=3; n=9.

FIG. 5 shows vardenafil, tamoxifen and their combination ameliorate penile fibrosis in an animal model for PD. FIG. 5A shows ΔICP measurement for different stimulation voltages in all the treatment groups. Intracavernous pressure (ICP) change from baseline to peak ICP (ΔICP). Data plotted as mean±SEM, N=7. FIG. 5B shows mRNA levels of α-SMA (ACTA2) were determined using the 2^{-ΔΔCt} method. Data points plotted as mean±SEM, N=4. *P<0.05 vs TGF-β1 injected group. mRNA levels of Col I were determined using the 2^{-ΔΔCt} method. Data points plotted as mean±SEM, N=4. *P<0.05 vs TGF-β1 injected group. FIG. 5C shows mRNA levels of Col III were determined using the 2^{-ΔΔCt} method. Data points plotted as mean±SEM, N=4. *P<0.05 vs TGF-β1 injected group. FIG. 5D shows mRNA levels of Col V determined using the 2^{-ΔΔCt} method. Data points plotted as mean±SEM, N=4. *P<0.05 vs TGF-β1 injected group. FIG. 5E shows mRNA levels of elastin determined using the 2^{-ΔΔCt} method. Data points plotted as mean±SEM, N=4. *P<0.05 vs TGF-β1 injected group or in between groups. FIG. 5F shows western blot quantification of α-SMA. Data shown as fold change between injected and uninjected side of the penis. Data points plotted as mean±SEM, N=3. *P<0.05 vs TGF-β1 injected group or in between groups. FIG. 5G shows western blot quantification of α-SMA/Col I ratio. Data shown as ratio of fold change between injected and uninjected side of the penis for α-SMA and Col I. Data points plotted as mean±SEM, N=3. *P<0.05 vs TGF-β1 injected group or in between groups. FIG. 5H shows western blot quantification of α-SMA/Col III ratio. Data shown as ratio of fold change between injected and uninjected side of the penis for α-SMA and Col III. Data points plotted as mean±SEM, N=3. *P<0.05 vs TGF-β1 injected group or in between groups. FIG. 5I shows western blot quantification of Col 1/Col III ratio. Data shown as ratio of fold change between injected and uninjected side of the penis for Col I and Col III. Data points plotted as mean±SEM, N=3. *P<0.05 vs TGF-β1 injected group or in between groups. TGF-β1=TGF-β1 injected group, vehicle=vehicle injected group, tamoxifen=TGF-β1 injected+tamoxifen treatment group, vardenafil=TGF-β1 injected+vardenafil treatment group, combined=TGF-β1 injected plus combined treatment group.

FIG. 6 shows immunohistochemical staining for different treatment groups. Representative images of Masson's Trichrome staining and H&E staining in whole sections of rat penis for: (6A) TGF-β1 injected group, left: Masson's Trichrome staining for injected side (4× magnification), right: H&E staining for injected side (4× magnification). (6B) TGF-β1 injected group treated with vardenafil, left: Masson's Trichrome staining for injected side (4× magnification), right: H&E staining for injected side (4× magnification). (6C) TGF-β1 injected group treated with tamoxifen, left: Masson's Trichrome staining for injected side (4× magnification), right: H&E staining for injected side (4× magnification). (6D) TGF-β1 injected group treated with combination of vardenafil and tamoxifen, left: Masson's Trichrome staining for injected side (4× magnification), right: H&E staining for injected side (4× magnification). Black arrow indicates smooth muscle. Orange box indicates collagenous fibrotic plaque. Blue arrows indicate nuclei (cellular infiltration due to inflammation).

### DETAILED DESCRIPTION

This invention relates to the reduction, inhibition or amelioration of fibrosis and the treatment of fibrotic conditions in patients using a combination of a phosphodiesterase type 5 inhibitor (PDE5i) and a selective oestrogen receptor modulator (SERM).

Fibrosis is the formation or development of excess fibrous connective tissue as a result of excess deposition of extracellular matrix (ECM) components, such as collagen and fibronectin. Fibrous connective tissue may for example be comprise extracellular matrix (ECM) with a high collagen content. The collagen in fibrous connective tissue may be provided in strands or fibres, which may be arranged irregularly or aligned. The ECM of fibrous connective tissue may also include glycosaminoglycans. Excess fibrous connective tissue may be an amount of connective tissue at a given location (e.g. a given tissue or organ, or part of a given tissue or organ) which is greater than the amount of connective tissue present at that location in the absence of fibrosis, e.g. under normal, non-pathological conditions. An excess deposition of extracellular matrix components may be a level of deposition of one or more extracellular matrix components which is greater than the level of deposition in the absence of fibrosis, e.g. under normal, non-pathological conditions.

The cellular and molecular mechanisms of fibrosis are described in Wynn, J. Pathol. 5 (2008) 214(2): 199-210, and Wynn and Ramalingam, Nature Medicine (2012) 18:1028-1040. The main cellular effectors of fibrosis are myofibroblasts, which produce a collagen-rich extracellular matrix. In response to tissue injury, damaged cells and leukocytes produce pro-fibrotic factors such as TGFβ, IL-13 and PDGF, which transform fibroblasts to αSMA-expressing myofibroblasts, and recruit myofibroblasts to the site of injury. Myofibroblasts produce a large amount of extracellular matrix and are important mediators in aiding contracture and closure of the wound. However, under conditions of persistent infection or during chronic inflammation there can be over activation and recruitment of myofibroblasts, and thus over-production of extracellular matrix components, resulting in the formation of excess fibrous connective tissue. In some embodiments, fibrosis may be characterised by myofibroblast transformation (i.e. the activation of fibroblasts to myoblasts).

A combination of a PDE5 inhibitor and a SERM as described herein may be useful in inhibiting myofibroblast transformation and/or the formation of excess fibrous connective tissue. This may be useful in inhibiting the process of fibrosis and reducing or preventing the formation of new fibrotic plaques.

Fibrotic conditions are pathological disorders that are characterized by the occurrence of fibrosis.

Fibrosis can occur in many tissues of the body. For example, fibrosis can occur in the liver (e.g. cirrhosis), lungs, kidney, heart, blood vessels, eye, skin, pancreas, intestine, brain, and bone marrow. Fibrosis may also occur in multiple organs at once.

Fibrotic conditions may involve an organ of the gastrointestinal system, e.g._of the liver, small intestine, large intestine, or pancreas, an organ of the respiratory system, e.g. the lungs, an organ of the cardiovascular system, e.g. of the heart or blood vessels, the skin, an organ of the nervous system, e.g. the brain, an organ of the urinary system, e.g. the kidneys, and/or an organ of the musculoskeletal system, e.g. muscle tissue.

Examples of fibrotic conditions that may be treated as described herein may include respiratory conditions such as pulmonary fibrosis, idiopathic pulmonary fibrosis, progressive massive fibrosis of the lung, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, primary biliary cirrhosis (PBC), schistosomal liver disease, liver cirrhosis; cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Chron's disease, microscopic colitis and primary sclerosing cholangitis (PSC); skin conditions such as scleroderma, scar formation, including burn scar formation, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease. Fabry's disease, diabetic nephropathy, chronic glomerulonephritis, nephritis associated with systemic lupus); progressive systemic sclerosis (PSS); chronic graft versus host disease; diseases of the eye such as Grave's ophthairnopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, pterygium; arthritis; fibrotic pre-neoplastic and fibrotic neoplastic disease; and fibrosis induced by chemical or environmental insult (e.g., cancer chemotherapy, pesticides, radiation/cancer radiotherapy).

The fibrotic condition may have a known trigger, such as skin burns and post-surgical fibrosis

In some preferred embodiments, the fibrotic condition may be Peyronie's disease (PD). An individual suitable for treatment as described herein may for example have acute/active phase PD.

A phosphodiesterase type 5 inhibitor (PDE5 inhibitor) is a compound that reduces or inhibits the activity cGMP-specific phosphodiesterase type 5. cGMP-specific phosphodiesterase type 5 specifically hydrolyzes intracellular messenger cGMP to inactive 5′-GMP (PDE5A; EC3.3.4.17; Gene ID 8654). The amino acid sequence of isoforms 1 to 3 of PDE5A are available from public databases under the accession numbers NP_001074.2; NP_236914.2 and NP_246273.2.

PDE5 inhibitors are known in the art for example for use in the treatment of erectile dysfunction and pulmonary hypertension and lower urinary tract symptoms. A PDE5 inhibitor for use in the treatment of a fibrotic condition as described herein may be selected from the group consisting of avanafil, lodenafil, mirodenafil, udenafil, benzamidenafil, dasantafil, sildenafil, vardenafil and tadalafil.

In some preferred embodiments, the PDE5 inhibitor may be vardenafil.

Selection estrogen receptor modulators (SERMs) are competitive partial agonists of the estrogen receptor (ER) and produce a range of estrogenic and anti-estrogenic effects in different tissues. ER is a dimer composed of ESR1 and ESR2 sub-units. The amino acid sequence of ESR1 (Gene ID 2099) isoforms 1-4 are available from public databases under the accession numbers NP_000116.2; NP_001278159.1, NP_001278170.1 and NP_001315029.1, respectively. The amino acid sequence of ESR2 (Gene ID 2100) isoforms 1-4 are available from public databases under the accession numbers NP_001035365.1; NP_001201831.1; NP_001258805.1; NP_001258806.1; NP_001278641.1; and NP_001278652.1, respectively.

SERMs are known in the art for example for use in the treatment of estrogen related disorders, such as breast cancer, ovulatory dysfunction and post-menopausal osteoporosis. SERM may be selected from the group consisting of tamoxifen, arzoxifene (LY353381), tormilene bazedoxifene (TSE-424), lasofoxifene (CP-336156), ospemifene, and raloxifene.

In some preferred embodiments, the SERM may be tamoxifen or raloxifene.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

The combination of a PDE5 inhibitor and a SERM is shown herein to be useful in the reduction, inhibition or amelioration of fibrosis and the treatment of fibrotic conditions in an individual.

An individual with a fibrotic condition may display at least one identifiable sign, symptom, or laboratory finding that is sufficient to make a diagnosis of the fibrotic condition in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001.

Treatment may be any treatment or therapy, whether of a human or an animal (e.g. in veterinary applications), in which a desired therapeutic effect is achieved, for example, the inhibition or delay of the formation of fibrotic plaques or the onset or progress of the fibrotic condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or individual beyond that expected in the absence of treatment.

Treatment as described herein may include prophylactic treatment (i.e. prophylaxis) i.e. the individual being treated may not have or may not be diagnosed as having a fibrotic condition at the time of treatment. For example, an individual susceptible to or at risk of the occurrence or re-occurrence of a fibrotic condition may be treated as described herein. Such treatment may prevent or delay the occurrence or re-occurrence of fibrotic condition in the individual or reduce its symptoms or severity after occurrence or re-occurrence. In some embodiments, the individual may have been previously identified as having increased susceptibility or risk of fibrotic condition compared to the general population or a method may comprise identifying an individual who has increased susceptibility or risk of fibrotic condition. Prophylactic or preventative treatment may be preferred in some embodiments.

While it is possible for PDE5 inhibitor and the SERM to be administered to the individual alone, it is preferable to present the compounds in the same or separate pharmaceutical compositions or formulations.

A pharmaceutical composition may comprise, in addition to the PDE5 inhibitor and/or the SERM, one or more pharmaceutically acceptable carriers, adjuvants, excipients, diluents, fillers, buffers, stabilisers, preservatives, lubricants, or other materials well-known to those skilled in the art. Such materials should be non-toxic and should not interfere with the efficacy of the active compound. The precise nature of the carrier or other material will depend on the route of administration, which may be by bolus, infusion, injection or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The formulations may conveniently be presented in unit dosage form and may be prepared by any methods well-known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The PDE5 inhibitor, SERM or pharmaceutical composition may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be by the oral route, although other routes such as intraperitoneal, subcutaneous, dermal or transdermal, intravenous, nasal, intramuscular or other convenient routes are not excluded. For example, the compounds may be applied dermally to prevent, reduce or treat scarring, including burn scarring. The pharmaceutical compositions comprising the active compounds may be formulated in a dosage unit formulation that is appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, ascorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Optionally, other therapeutic or prophylactic agents may be included in the pharmaceutical composition or formulation.

PDE5i and SERM may be administered as described herein to a subject or individual in therapeutically-effective amounts.

The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio.

The appropriate dosage of an active compound may vary from individual to individual. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the individual. The amount of active compounds and route of administration will ultimately be at the discretion of the physician, although generally the dosage will be to achieve therapeutic plasma concentrations of the active compound without causing substantial harmful or deleterious side-effects.

In general, a suitable dose of the active compound is in the range of about 100 μg to about 400 mg per kilogram body weight of the subject per day, preferably 200 μg to about 200 mg per kilogram body weight of the subject per day. Where the active compound is a salt, an ester, prodrug, or the like, the amount administered is calculated on the basis of the parent compound and so the actual weight to be used is increased proportionately.

Suitable clinical doses of SERMs and PDE5 inhibitors may be readily determined. For example, vardenifil may be administered at 20 mg daily and tamoxifen may be administered at 20 mg twice daily.

Administration in vivo can be effected in one dose, continuously or intermittently (e.g., in divided doses at appropriate intervals).

Methods of determining the most effective means and dosage of administration are well known in the art and will vary with the formulation used for therapy, the purpose of the therapy, the target cell being treated, and the subject being treated. Single or multiple administrations can be carried out with the dose level and pattern being selected by the physician.

Multiple doses of the SERM and PDE5 inhibitor may be administered, for example 2, 3, 4, 5 or more than 5 doses may be administered. The administration of the SERM and PDE5 inhibitor may continue for sustained periods of time. For example treatment with the PDE5 inhibitor and SERM may be continued for at least 1 week, at least 2 weeks, at least 3 weeks, at least 1 month or at least 2 months. Treatment with the PDE5 inhibitor and SERM may be continued for as long as is necessary to reduce symptoms or cure the condition.

The SERM and PDE5 inhibitor may be administered alone or in combination with other treatments, either simultaneously or sequentially dependent upon the individual circumstances. For example, a SERM and PDE5 inhibitor as described herein may be administered in combination with one or more additional active compounds, including anti-fibrotic compounds, such as pirfenidone or nintedanib.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### Experimental

### Materials and Methods

### Acquisition of Tunica Albuginea Samples and Isolation of Fibroblasts

Tunica albuginea tissue samples were collected from patients undergoing surgery at University College London Hospital (UCLH), London, UK for penile cancer or PD. All patients gave fully informed written consent to the study. The study was approved by independent research ethics committees (NRES Committee East of England 12/EE/0170 and NRES Committee North of Scotland 15/NS/0051). PD plaque tissue was obtained from patients with chronic PD undergoing a Lue procedure (plaque incision and grafting). Plaque tissue would have otherwise been discarded. Non-plaque TA was obtained from PD patients undergoing a Nesbit procedure whereby non-fibrotic TA tissue was excised from the opposite side of the plaque. TA tissue samples from patients with penile cancer were taken from the proximal side away from the tumour, with the tumour showing negative margins on histological examination.

Tissue samples were carefully dissected to ensure that all cavernosal tissue was removed from the tunica albuginea (TA). To establish fibroblast cultures, TA fragments were seeded in 6-well tissue culture plates (Nunc, Fisher Scientific, UK) as described previously16. The tissue pieces were incubated in DMEM-F12 (GIBCO, Invitrogen, UK) containing 10% FCS (Fisher Scientific, UK) and 1% penicillin-streptomycin (GIBCO, Invitrogen, UK) at 37° C., 5% CO2 for 5-7 days. Tissue fragments were carefully removed using forceps upon outgrowth of cells. Passages 2 to 4 were used for the rest of the experiments.

### In Cell ELISA (ICE)

Cells were seeded onto 96 well optical flat bottom black microplates (Nunc, Fisher Scientific, UK) at 5×103 cells per well. After overnight attachment they were incubated with or without 10 ng/ml TGF-β1 for 72 hours. The cells were then fixed using 4% paraformaldehyde, and blocked with 10% donkey serum and 0.1% Triton X-100 in PBS. The cells were then incubated with anti-α-SMA antibody (1:3,000; Sigma Aldrich, UK) for 2 h. Afterwards cells were incubated with donkey anti-mouse secondary antibody conjugated to an infrared dye which emits at 800 nm (1:500; IRdye 800CW; Li-Cor, UK) and a nuclear counterstain at that emits at 700 nm (1:1,000; DRAQS, Biostatus, UK) for 1 h. The plate was scanned using an infrared imaging system (Odyssey CLx imager, LI-COR, UK) at both the 700 nm and 800 nm wavelengths.

### Collagen Gel Contraction Assay

Cell Contraction Assay (Cell Biolabs Inc, CBA-201) was used according to the manufacturer's instructions. Briefly, 10,000 cells/well were mixed with collagen solution and DMEM with or without 10 ng/ml TGF-β1 and then plated into 96 well plates. Cultures were incubated for 3 days at 37° C., 5% CO2 and lattices were released from the walls of the wells using a sterile spatula or needle. Contraction of the collagen lattices was observed for 8 h and documented using a digital camera (Canon Digital IXUS 55, 5.0 mega pixels). Images were analysed using ImageJ software by measuring the surface area of the contracting lattice. Contraction was calculated as percentage of the surface of the unreleased lattice. Data is shown as percentage of maximum contraction of vehicle control.

### ECM Production Assay

Cells were seeded onto 96 well optical flat bottom black microplates (Nunc, Fisher Scientific, UK) at 5×103 cells per well. After overnight attachment, they were stimulated with or without 10 ng/ml TGF-β1 and/or compounds for 7 days. DRAQS in PBS (1:1,000) was added and cells were incubated for 5 min at 37° C., 5% CO2 before scanning the plate to obtain nuclear staining. Cells were then lysed using ammonium hydroxide as described previously¹⁷ and ECM was fixed using a solution containing 50% methanol and 7.5% acetic acid for 1 h at −20° C. Afterwards ECM was stained either with Coomassie Blue (total ECM) overnight at 4° C. or with primary antibodies (collagen I, abcam; collagen III, Millipore; collagen V, abcam: fibronectin, Millipore) at 1:1000 for 1 h on a shaker, followed by incubation with secondary antibody and scanning the plate using an infrared imaging system (Odyssey CLx imager, LI-COR, UK) at both the 700 nm and 800 nm wavelengths. Results were normalized to the cell number before lysis.

### Animal Treatment

The animal model for PD was first described by El-Sakka and Lue¹⁸ and modified by Bivalacqua and Hellstrom¹⁹. Male Sprague-Dawley rats (10-12 weeks old) were housed in a regulated environment with a 12-hour light/dark cycle in a standard experimental laboratory. The animals had free access to food and water ad libitum. Fifty male Sprague-Dawley rats were divided into 5 groups: A: sham (injection of vehicle citrate buffer), B: TGF-β1 injection (1 μg in 100 μl citrate buffer (10 mM, Sigma-Aldrich, UK)), C: TGF-β1 injection+tamoxifen (5 mg/kg/day; i.p.), D: TGF-β1 injection+vardenafil (1.5 mg/kg/day; drinking water), E: TGF-β1 injection+tamoxifen+vardenafil. Treatment was initiated at the day after injury and continued for 5 weeks followed by a 48 hours wash-out period.

### Assessment of Erectile Function

At the end of wash off period, under ketamine (100 mg/kg) and xylazine (10 mg/kg) anaesthesia, the major pelvic ganglion (MPG) and cavernous nerve (CN) were exposed bilaterally via midline laparotomy. A 25 G butterfly needle, filled with 250 U/ml heparin solution, was inserted into the proximal left corpus cavernosum and connected to a pressure transducer for ICP measurement. The ICP was recorded at a rate of 25 samples per second. A bipolar stainless-steel hook electrode was used to stimulate the CN directly via a signal generator and custom-built constant-current amplifier generating monophasic rectangular pulses with stimuli of 5, 7.5, 10, and 15V. Depending on the anatomical positioning and accessibility of the nerve, the stimulations were performed on either the left or right MPG/CN. The maximal amplitude of ICP during nerve electrostimulation was calculated from baseline value and included for statistical analysis in each animal. Systemic blood pressure was recorded by inserting a PE-50 polyethylene tubing into the right common carotid artery. After functional testing, animals were euthanized by cervical dislocation. Following these measurements, the penis was harvested for histological, molecular and transcriptional analysis.

qPCR.

The High-Capacity cDNA Reverse Transcription Kit (Applied Biosystems) was used according to the manufacturer's instructions to transcribe RNA to cDNA. One μg in 10 μl were added to 10 μl of the master mix for reverse transcription for a total reaction volume of 20 μl. After reverse transcription the cDNA was diluted 1:10 for qPCR. The Applied Biosystems™ TaqMan™ Fast Advanced Master Mix was used for qPCR. Gene specific primer pairs for GAPDH, beta-actin, alpha smooth muscle actin, elastin, and collagens I, III, and V were purchased from Applied Biosystems. StepOnePlus™ Real-Time PCR System and software were used for the experiments and data analysis. Data was analysed using the 2−ΔΔCt method for relative quantifications.

### Western Blot

20-30 μg of protein was mixed 1:1 with 2× laemmli buffer (Bio-Rad) under reducing or non-reducing conditions and heat denatured at 95° C. for 4 minutes. Samples were loaded onto an Any kD™ Mini-PROTEAN® TGX™ Precast Protein Gel (Bio-Rad) along with 5 μl of a protein ladder (Bio-Rad). After the gel electrophoresis the transfer onto a methanol activated PVDF membrane (Bio-Rad) was achieved by wet blotting for 1 hr at 350 mA. Membranes were washed before blocking the unspecific binding with 10% (w/v) non-fat dried milk (NFDM, Marvel) in 0.1% TBS-T for 1 h. Primary antibodies were diluted in 5% NFDM in 0.1% TBS-T and incubated 0/N at 4° C. on a shaker. Subsequently membranes were washed 4× with 0.1% TBST and blocked again. After this, the secondary antibodies were added in a dilution of 1:3,000 in 5% NFDM with 0.1% TBS-T and incubated for 1 h on a shaker in the dark. Four minutes washes with 0.1% TBS-T for 5 minutes were followed with a 5 minutes incubation of an enhancer solution (Supersignal west dura, Thermo Fisher). Blots were visualized using a Syngene documentation system and Genesys Software. Statistical Analysis Data analysis was performed using Microsoft Excel 2013 or Graph Pad Prism 7 software. Statistical significance, unless otherwise stated, was calculated using one or two-way ANOVA and Student's t-test for unpaired means (two-sided). Prior to performing this calculation, Ftest of equality of variances was performed, to ensure equal variance could be assumed when performing Student's t-test. A P value less than 0.05 was considered statistically significant. All in vitro experiments were performed in at least triplicate of three experiments (n=9) using samples from 3 patients (N=3). 8 rats were used in each group in in vivo experiments.

### Dose Selection:

In this study, the dose selection for tamoxifen and vardenafil was based on the body surface area normalization method42, yielding an animal dose equivalent to the doses used in the clinics. Using this method, the animal equivalent dose for tamoxifen was calculated from the dose used in the clinical study that indicated an effect for tamoxifen in the early stage of PD33, in which the patients were given 20 mg of tamoxifen twice a day. The calculated animal equivalent dosage for rats would be 4.2 mg/kg/day of tamoxifen which is also within the ethically acceptable limits for tamoxifen treatment in this species. For vardenafil the highest dose used in humans was selected as a starting point. 20 mg a day in humans correspond to a dose of 2 mg/kg/day in rats which is higher than the dose of 1.5 mg/kg/day that was utilized in this study, limited by the water solubility of the drug. Increasing the concentration of vardenafil would have meant further acidifying the drinking water, which would have had a negative effect on drinking behaviour and consequently animal welfare. No effect on drinking behaviour could be observed with the protocol chosen for this study. As a result of these calculations and observations, doses to be used in the animal model were finalised as 5 mg/kg/day and 1.5 mg/kg/day for tamoxifen and vardenafil respectively.

### Results

### Development and Validation of the Phenotypic Assay:

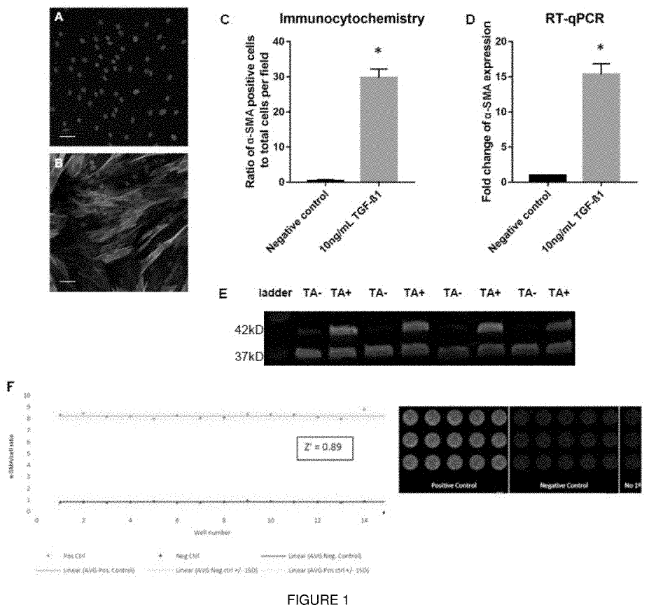

We have isolated primary fibroblasts from the plaque and non-plaque TA of patients with PD. We also isolated primary fibroblasts from TA of patients with penile cancer as non-fibrotic controls. The primary fibroblasts were similar in morphology and function (as shown by their response to TGF-β1) in all three groups: fibroblasts derived from plaque of PD patients, fibroblasts derived from non-plaque TA of PD patients and fibroblasts derived from TA of patients with penile cancer. Based on this similarity, we have utilised fibroblasts derived from non-plaque TA of PD patients throughout these experiments since they would be more representative of fibroblasts that have not been exposed to pro-fibrotic environment. The fibroblast identity of the TA-derived cells was validated. Upon exposure to TGF-β1 (10 ng/ml for 72 hrs), we observed a significant 8-fold increase in α-SMA expression in both mRNA and protein levels in TA-derived fibroblasts (FIG. 1). We then developed a phenotypic screening assay in a 96-well plate format using in-cell ELISA (ICE) where the cell viability and α-SMA protein expression can be simultaneously measured in a reproducible manner (Z′=0.89; FIG. 1). The assay was further validated using vehicle control and a TGF-β1 receptor antagonist (SB505124) where the cells remained viable in up to 1% DMSO and SB505124 inhibited TGF-β1-induced myofibroblasts transformation in a concentration-dependent manner (1050=0.6 μM).

### Hit Identification:

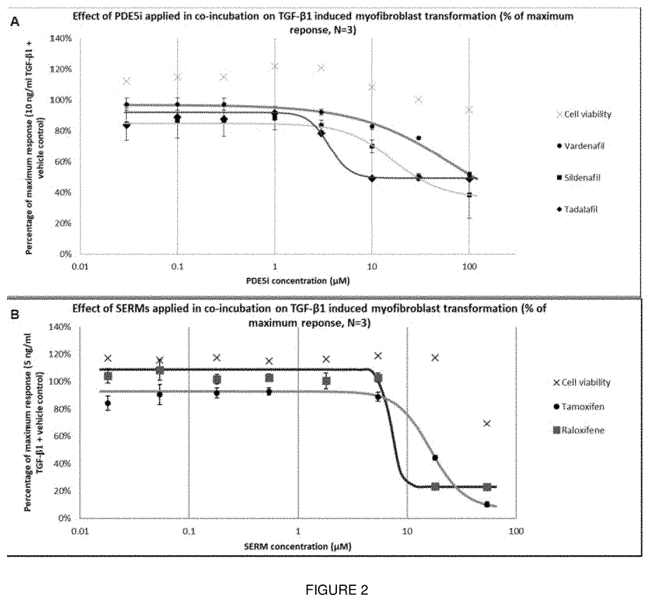

We then tested twenty-one compounds/drugs (Table 1) which have been suggested to be efficacious in PD based on in vitro and in vivo studies and/or early-phase clinical studies. Out of these 21 drugs only 2 classes, selective oestrogen receptor modulators (SERMs) and PDE5 inhibitors (PDE5i), showed significant inhibition on myofibroblast transformation. Full concentration response curves were constructed for these hits, which yielded inverse sigmoid curves with an upper and lower plateau without affecting the cell viability (FIG. 2). The following molecules where used to investigate the two classes of drugs: vardenafil, sildenafil and tadalafil as PDE5i (IC₅₀=30 μM, 15 μM and 3.5 μM respectively) and tamoxifen and raloxifene as SERMs (IC₅₀=11.9 μM and 7 μM respectively). When a PDE5i, vardenafil and a SERM, tamoxifen were tested in combination, a synergy between the two drugs became apparent (the observed inhibition was greater than the arithmetic sum of each) (FIG. 2, Table 2).

### Functional Assays:

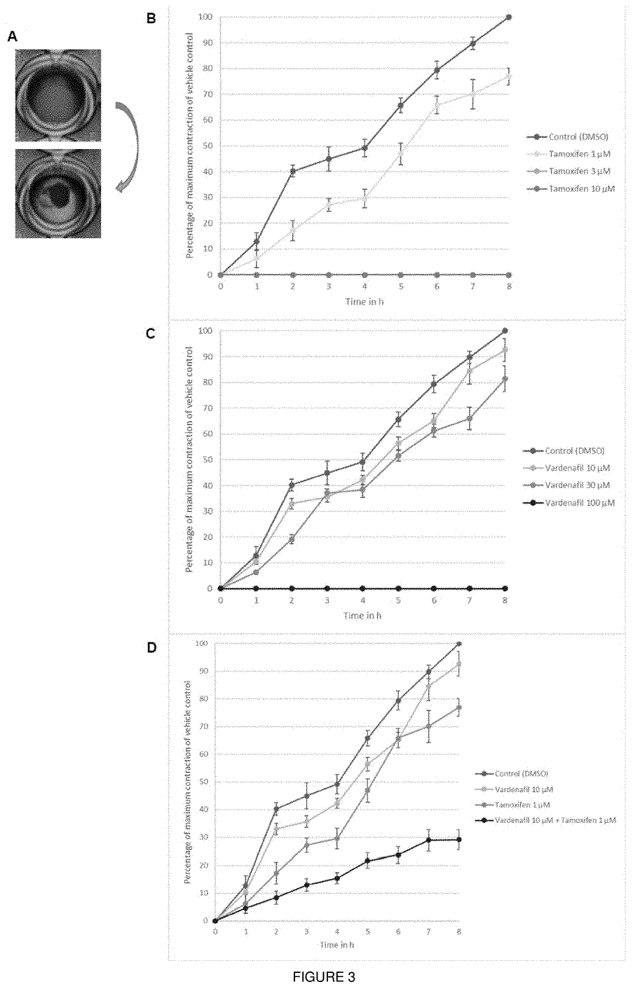

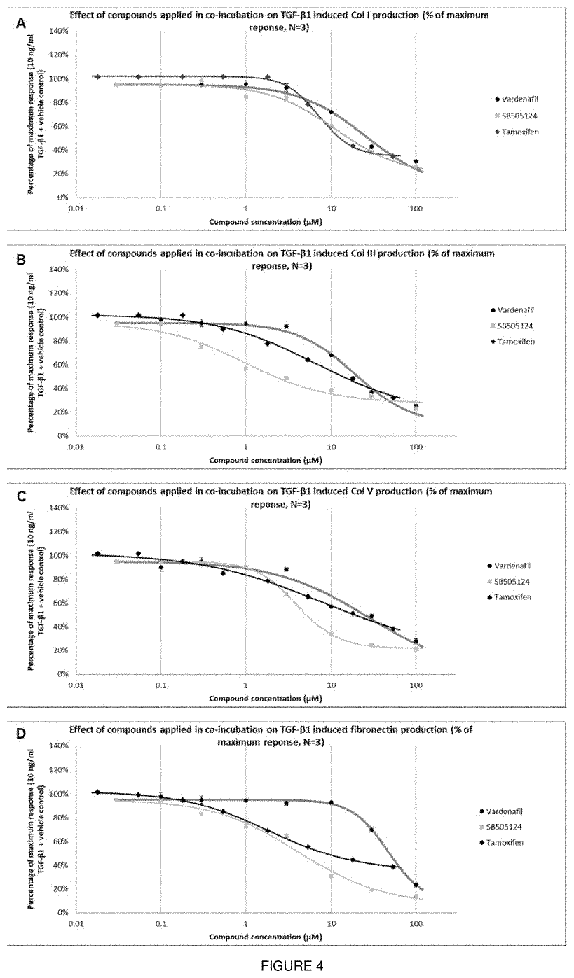

The two classes of drugs were then tested in functional assays. Firstly, we tested their efficacy in inhibiting collagen gel contraction as a measure of myofibroblast contractility; a characteristic function of myofibroblasts, which separates them from fibroblasts. Collagen gels were loaded with fibroblasts and contraction was measured after stimulation with TGF-β1 as described before^{20,21}. Both vardenafil and tamoxifen inhibited TGF-β1-induced contraction of collagen at concentrations of 10 μM and 1 μM respectively (FIG. 3). Secondly, we tested the drugs' efficacy in inhibiting ECM protein production, which is again one of the critical characteristic functions of myofibroblasts. Again, both vardenafil and tamoxifen inhibited TGF-β1-induced ECM protein production (collagens I, III, V and fibronectin) (IC₅₀=23 μM, 17 μM, 23 μM, and 44 μM for vardenafil and 7 μM, 5.3 μM, 6.7 μM and 1.6 μM for tamoxifen, respectively; FIG. 4).

### In Vivo Testing of the Two Hits:

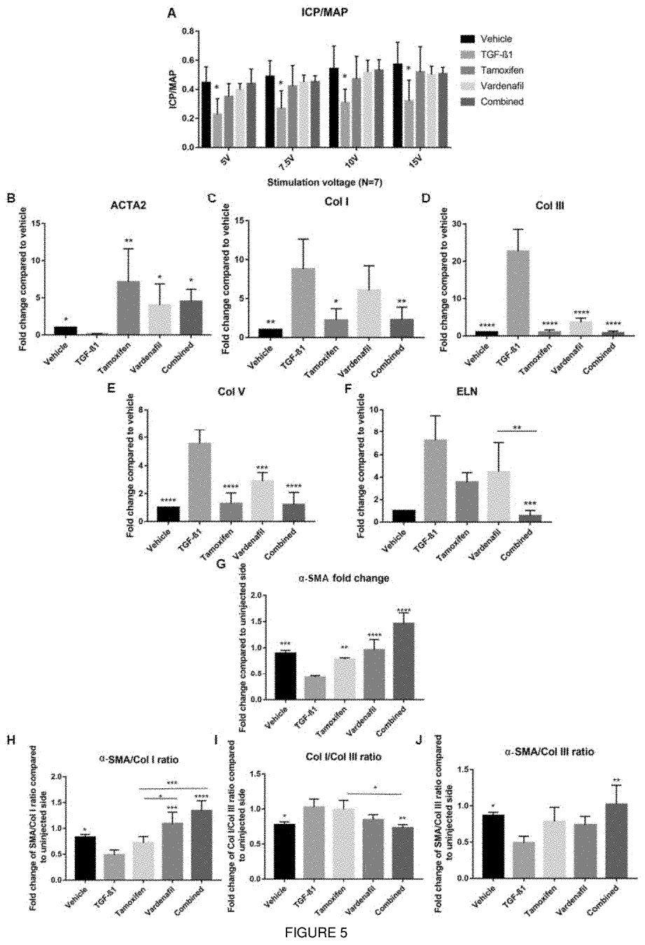

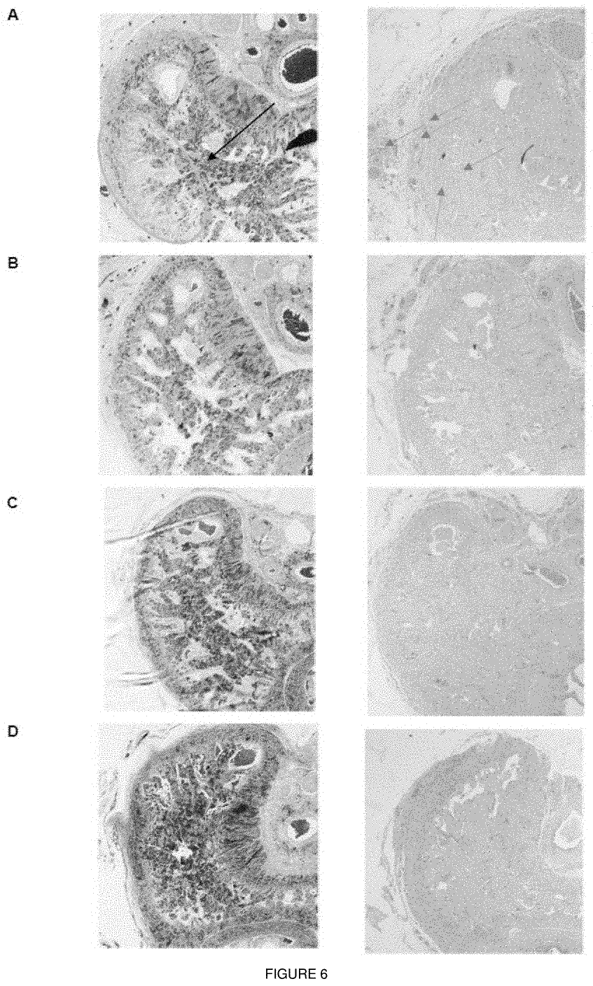

To elucidate whether the drugs could also prevent fibrosis in vivo, they were taken further to be tested in an animal model for PD. Five weeks after TGF-β1 injection into the rat TA, with or without treatment with vardenafil, tamoxifen or their combination, the rats were subjected to erectile function measurement (intracavernous pressure measurement; ICP) before harvesting the penis for molecular analysis. The ICP measurement revealed that TGF-β1 injection led to a decrease in erectile function by 55% which was prevented in all treatment groups. Treatment groups showed no significant differences in ICP compared to the vehicle injected group (FIG. 5). Subsequent mRNA expression analysis of the penile tissue harvested from the rats showed that expression of collagens I, III, and V were significantly upregulated in the TGF-β1 injection group, but not in the vehicle or treated groups. Treatment with vardenafil or tamoxifen therefore prevented upregulation of TGF-β1-induced increase in collagen expression. Interestingly, the combination of vardenafil and tamoxifen acted synergistically on the down-regulation of elastin (FIG. 5). The formation of fibrosis in response to TGF-β1 injection into the penis was further confirmed by measuring α-SMA in the corpus cavernosum (as a measure of loss of smooth muscle mass due to fibrotic tissue) using Western blot and immunohistochemistry. The results showed a significant loss of smooth muscle which was prevented in the treatment groups (FIG. 5). Furthermore, immunohistochemistry using H&E and Masson's Trichrome staining showed increased infiltration of inflammatory cells, formation of fibrosis and loss of smooth muscle in the TGF-β1 injected group; effects which were prevented in the treatment groups (FIG. 6).

In contrast to the single-target approach, the phenotypic screening seeks to find compounds that target a phenotype rather than a single molecular target. In this case we have chosen transformation of fibroblasts to myofibroblasts as the target phenotype and developed an assay which can quantify inhibition of myofibroblast transformation in a reproducible manner. Using this assay, we then tested 21 compounds/drugs that have been suggested as potentially anti-fibrotic agents. Among this cohort, only two groups were able to inhibit myofibroblast transformation: PDE5i and SERMs. When applied together, the two classes showed synergistic activity both in vitro and in vivo.

To our knowledge this is the first study to show a synergy between PDE5i and SERMs. PDE5i have previously been suggested to be effective anti-fibrotic agents in vitro using TA-derived fibroblasts²². This was further confirmed in an animal model for PD with long term vardenafil treatmem^{23,24.} Furthermore, in vivo studies led to PDE5i being proposed as a potential treatment for other fibrotic disorders such as muscle fibrosis in a Duchenne muscular dystrophy mouse model₂₅ and for prevention of cardiac fibrosis and its underlying cardiac fibroblast activation²⁶. Tamoxifen has been shown to be effective in animal models for renal tubulo-interstitial fibrosis and peri-portal hepatic fibrosis^{27,28.} The anti-myofibroblast effect has also been reported in models utilizing TGF-β1-mediated activation of primary human dermal and breast fibroblasts²⁹. Previous research suggested that the effect of tamoxifen on fibroblast-mediated collagen contraction is either due to downregulation of TGF-β2³⁰ or a change in morphology of fibroblasts³¹. Oestradiol has been shown to inhibit transformation of TA-derived fibroblast to myofibroblasts₃₂. However, there is no previous study that has investigated the effect of tamoxifen in an animal model of PD.

Previous clinical studies have shown mixed results using PDE5i and SERMs³³⁻³⁷. An open label single-arm study with tamoxifen showed a positive effect₃₃ while a later placebo-controlled study showed no effect with tamoxifen³⁴. However, the open label study noted that tamoxifen showed some improvement in patients with early PD³³; while all of the patients in the latter study were in the late phase of the disease³⁴. Similarly, in studies with PDE5i, the results have been mixed³⁶⁻³⁷. This is not surprising since the drugs were tested on patients with established plaque-fibrotic tissue. Our results suggest that PDE5i or SERMs inhibit myofibroblast transformation and ECM production; they would not be able to reverse the established/preformed fibrosis. We are therefore proposing that the combination of PDE5i and SERMs will inhibit myofibroblast transformation, hence new fibrosis formation and prevent new plaque formation. We believe that more and more men are presenting at early stages of the disease as there are now better information resources and access to healthcare to have symptoms investigated. Indeed, others have reported that 30-40% of patients present with progressing deformity³⁸⁻⁴⁰. This drug combination may be more effective in patients in the acute phase where penile pain or the onset of a nodule would be an indication for referral. This will be an area for patient and primary care education. The doses of vardenafil and tamoxifen used in our animal model are representative of their clinical doses; tamoxifen 20 mg twice daily and vardenafil 20 mg daily.

In summary, this is the first study to demonstrate a synergistic anti-fibrotic effect of a combination of PDE5i and SERMs in in vitro and in vivo disease models. Future prospective clinical trials using a combination of these drugs should be considered during the active phase of PD, given the early evidence of benefit in both in vitro and in vivo models. The data herein is indicative that the combination will be more efficacious than using either of the drugs as a monotherapy. These results are likely to lead to further research into the interaction between the two pathways and development of novel therapeutic approaches for the prevention and/or treatment of other fibrotic diseases.

Table 1 below shows the compounds and drugs tested with the phenotypic screening assay. Full concentration response curve with IC₅₀ values were only constructed for preliminary hits in the screening campaign. Hits highlighted in red

Table 2 shows synergistic effect of vardenafil in co-incubation with tamoxifen on TGF-β1-induced myofibroblasts transformation. Cells derived from non-PD TA tissue were exposed to a range of concentrations of vardenafil between 0.03 and 100 μM in co-incubation with 10 ng/ml TGF-β1 for 72 hours. Additionally, 1 μM of tamoxifen was added, N=3. Statistical significance determined by using Holm-Sidak t-test, *P<0.05 vs predicted additive effect.

TABLE 2
Percentage of inhibition of TGF-β1 induced
myofibroblast transformation

| | | | | Vardenafil | Vardenafil |
|---|---|---|---|---|---|
| | | | | plus 1 μM | plus 1 μM |
| | | | | tamoxifen: | tamoxifen: |
| | | | | Predicted | Observed |
| | | Tamoxifen | Vardenafil | additive | synergistic |
| | | only | only | effect | effect |
| 0.3 | μM | 10 ± 3% | 3 ± 3% | 13 ± 5% | 22 ± 2% * |
| 1 | μM | 10 ± 2% | 10 ± 1% | 20 ± 3% | 23 ± 2% * |
| 3 | μM | 10 ± 3% | 8 ± 2% | 18 ± 4% | 24 ± 3% * |
| 10 | μM | 25 ± 3% | 17 ± 2% | 27 ± 3% | 29 ± 2% |
| 30 | μM | 80 ± 2 | 25 ± 1% | 35 ± 3% | 38 ± 1% * |
| 100 | μM | cell death | 48 ± 1% | 58 ± 2% | 55 ± 1% * |

### REFERENCES

1. Schwarzer U, et al. BJU Int 2001; 88:727-30 doi:10.1046/j.1464-4096.2001.02436.x.
2. Hellstrom W J G. Int J Impot Res 2003; 15:S91-.
3. Nelson C J, et al. J Sex Med 2008; 5:1985-90. doi:10.1111/J.1743-6109.2008.00895.X.
4. Egui Rojo M A, et al. Ther Adv Urol 2014; 6:192-7. doi:10.1177/1756287214537331.
5. Andrews H O et al. BJU Int 2002; 87:658-60. doi:10.1046/j.1464-
6. McAnulty R J. Int J Biochem Cell Biol 2007. doi:10.1016/j.biocel.2006.11.005.
7. Hinz B et al. Am J Pathol 2007; 170:1807-16. doi:10.2353/AJPATH.2007.070112.
8. Borthwick L A et al. Biochim Biophys Acta—Mol Basis Dis 2013; 1832:1049-60. doi:10.1016/J.BBADIS.2012.09.014.
9. McAnulty R J et al. Biochim Biophys Acta—Mol Cell Res 1991; 1091:231-5. doi:10.1016/0167-4889(91)90066-7.
10. Guyot C et al. Int J Biochem Cell Biol 2006; 38:135-51. doi:10.1016/J.BIOCEL.2005.08.021.
11. Ask K, Martin G E M, Kolb M, Gauldie J. doi:10.1513/pats.200602-021TK.
12. Nandhini T. J Pharm Sci Res 2014; 6:334-7.
13. Jalkut M et al. Curr Urol Rep 2004. doi:10.1007/s11934-004-0074-y.
14. Gelfand R A et al. J Sex Med 2015. doi:10.1111/jsm.12760.
15. Bollong M J, et al. Proc Natl Acad Sci USA 2017; 114:4679-84. doi:10.1073/pnas.1702750114.
16. Mateus M, et al. J Sex Med 2018. doi:10.1016/j.jsxm.2018.05.003.
17. Fisher M, et al. Matrix Biol 2009. doi:10.1016/j.matbio.2008.10.003.
18. El-Sakka A I, et al. J Urol 1997; 158:2284-90. doi:10.1016/S0022-5347(01)68236-3.
19. Bivalacqua T J, et al. J Urol 2000; 163:1992-8. doi:10.1016/50022-5347(05)67616-1.
20. Bell E et al. Proc Natl Acad Sci 1979. doi:10.1073/pnas.76.3.1274.
21. Dallon J C, et al. Wound Repair Regen 2008. doi:10.1111/j.1524-475X.2008.00392.x.
22. Valente E G A et al. Nitric Oxide—Biol Chem 2003. doi:10.1016/j.niox.2003.12.002.
23. Ferrini M G et al. BJU Int 2006. doi:10.1111/j.1464-410X.2006.05955.x.
24. Gonzalez-Cadavid N F, et al. Nat Rev Urol 2010. doi:10.1038/nrurol.2010.24.
25. Nio Y et al. n.d. doi:10.1096/fj.201700249R.
26. Gong W, et al. Front Med 2014. doi:10.1007/s11684-014-0378-3.
27. Kim D, et al. Nephrol Dial Transplant 2014. doi:10.1093/ndt/gfu240.
28. Ryu S H, et al. Liver Int 2009. doi:10.1111/j.1478-3231.2008.01811.x.
29. Carthy J M, et al. J Cell Physiol 2015. doi:10.1002/jcp.25049.
30. Kuhn M A et al. J Surg Res 2002; 103:146-52. doi:10.1006/JSRE.2001.6350.
31. Hu D et al. Br J Plast Surg 1998. doi:10.1054/bjps.1997.0100.
32. Jiang H S, et al. Int J Mol Med 2015; 36:801-7. doi:10.3892/ijmm.2015.2288.
33. Ralph D J, et al. Br J Urol 1992:648-51.
34. Teloken C, et al. J Urol 1999. doi:10.1016/50022-5347(05)68087-1.
35. Dell'Atti L. Urol Ann 2015; 7:345-9 doi:10.4103/0974-7796.152048.
36. Ozturk U et al. Irish J Med Sci (1971-) 2014; 183:449-53. doi:10.1007/s11845-013-1036-5.
37. Park T Y et al. World J Mens Health 2016; 34:40-6. doi:10.5534/wjmh.2016.34.1.40.
38. Kadioglu A et al. J Urol 2002; 168:1075-9. doi:10.1016/S0022-5347(05)64578-8.
39. Mulhall J P et al. J Urol 2006; 175:2115-8. doi:10.1016/S0022-5347(06)00270-9.
40. Berookhim B M et al. BJU Int 2014. doi:10.1111/bju.12346.
41. Stebbeds W et al. Asian J Androl 2014; 16:639. doi:10.4103/1008-682X.126395.
42. Reagan-Shaw S, et al. FASEB J 2007. doi:10.1096/fj.07-9574LSF.
43. Travers et al Circ Res 2016 118(6) 1021-1040

1. A method of treatment of a fibrotic condition comprising administering a phosphodiesterase type 5 (PDE5) inhibitor and a selective oestrogen receptor modulator (SERM) to an individual in need thereof.

2. The method according to claim 1, wherein the PDE5 inhibitor is selected from the group consisting of avanafil, lodenafil, mirodenafil, udenafil, benzamidenafil, dasantafil, sildenafil, vardenafil and tadalafil.

3. The method according to claim 1, wherein the PDE5 inhibitor is vardenafil.

4. The method according to claim 1, wherein the SERM is selected from the group consisting of arzoxifene (LY353381), toremifene, bazedoxifene (TSE-424), lasofoxifene (CP-336156), ospemifene, raloxifene, or tamoxifen.

5. The method according to claim 1, wherein the SERM is tamoxifen.

6. The method according to claim 1, wherein the fibrotic condition is a pulmonary, liver, heart, kidney, skin or brain fibrotic condition.

7. The method according to claim 6, wherein the pulmonary fibrotic condition is idiopathic pulmonary fibrosis or radiation induced lung injury.

8. The method according to claim 6, wherein the liver fibrotic condition is cirrhosis.

9. The method according to claim 6, wherein the heart fibrotic condition is atrial fibrosis or endomyocardial fibrosis.

10. The method according to claim 6, wherein the brain fibrotic condition is a glial scar.

11. The method according to claim 1, wherein the fibrotic condition is selected from the group consisting of arterial stiffness, arthrofibrosis, Crohn's disease, Dupuytren's contracture, keloid, mediastinal fibrosis, myelofibrosis, Peyronie's disease (PD), nephrogenic systemic fibrosis, progressive massive fibrosis, retroperitoneal fibrosis, burn scar, post-operative fibrosis of any organ, urethral fibrosis, diabetic nephropathy or scleroderma/systemic sclerosis.

12. The method according to claim 11, wherein the fibrotic condition is Peyronie's disease (PD).

13. The method according to claim 12, wherein the PD is active phase PD.
